(19) United States
(12) Patent Application Publication
(10) Pub. No.: US 2012/0153038 A1
(43) Pub. Date: Jun. 21, 2012

(54) SCENT-RELEASING APPARATUS AND METHOD OF MAKING SAME

(76) Inventor: Bryan Bruce Willert, Saint Louis, MO (US)

(21) Appl. No.: 12/969,116

(22) Filed: Dec. 15, 2010

(51) Int. Cl. A61L 9/12

(52) U.S. Cl. 239/34

(57) ABSTRACT

A scent-releasing apparatus is provided. The scent-releasing apparatus includes a housing having a base and a cap coupled to the base to define an interior space. At least one of the base and the cap includes a plurality of apertures for permitting ambient airflow into the interior space. The scent-releasing apparatus also has a dispenser positioned within the housing. The dispenser includes a blister and a gas-impermeable membrane layer removably coupled to the blister for covering a volume for containing a scent-releasing material, the gas-impermeable membrane layer configured to substantially seal the scent-releasing material from ambient air when in a sealed position. The gas-impermeable membrane layer includes a tab portion extending at least partially outside of the housing, the tab portion configured to prompt removal of the gas-impermeable membrane layer from the sealed position to an unsealed position in which the scent-releasing material is exposed to ambient air.

### BACKGROUND OF THE INVENTION

The subject matter described herein relates generally to a scent-releasing apparatus and, more particularly, to a scent-releasing apparatus for use in deodorizing and a method of making the same.

Many known deodorizing devices have a scent-releasing material that vaporizes in the presence of ambient air. For example, the scent-releasing material may include a liquid material that volatizes in the presence of ambient air, or a solid material that sublimes in the presence of ambient air. When deploying these deodorizing devices, the scent-releasing material is exposed to the ambient air to permit contact between the ambient air and the scent-releasing material, thereby causing the release of a deodorizing scent into the ambient air. These devices may then be placed in locations that the user desires to deodorize (e.g., a restroom, a basement, etc.).

When these devices are sold to the consumer, however, the scent-releasing material is substantially sealed in an air-tight manner to prevent premature exposure of the scent-releasing material to the ambient air (e.g., during shipment and storage). As such, when a user desires to deploy these devices, the user is often required to handle the devices in close proximity to the scent-releasing material in order to expose the scent-releasing material to the ambient air and initiate release of the deodorizing scent. However, handling the devices in such close proximity to the scent-releasing material can be undesirable.

As such, it would be useful to provide a scent-releasing device that can be more easily deployed without the user having to handle the device in such close proximity to the scent-releasing material.

### BRIEF DESCRIPTION OF THE INVENTION

In one aspect, a scent-releasing apparatus is provided. The scent-releasing apparatus includes a housing having a base and a cap coupled to the base to define an interior space, wherein at least one of the base and the cap includes a plurality of apertures for permitting ambient airflow into the interior space. The scent-releasing apparatus also has a dispenser positioned within the housing. The dispenser includes a blister and a gas-impermeable membrane layer removably coupled to the blister for covering a volume for containing a scent-releasing material. The gas-impermeable membrane layer is configured to substantially seal the scent-releasing material from ambient air when in a sealed position. The gas-impermeable membrane layer includes a tab portion extending at least partially outside of the housing. The tab portion is configured to prompt removal of the gas-impermeable membrane layer from the sealed position to an unsealed position in which the scent-releasing material is exposed to ambient air.

In another aspect, a housing for a scent-releasing apparatus having dispenser is provided. The dispenser defines a volume for containing a scent-releasing material that is substantially sealed from ambient air by a gas-impermeable membrane layer that is removable from a sealed position to an unsealed position via a tab portion to expose the scent-releasing material to the ambient air and release a scent into the ambient air. The housing includes a base and a cap configured to be coupled to the base to define an interior space of the housing, wherein at least one of the base and the cap has a plurality of apertures for permitting ambient airflow into the interior space. The housing is configured to position the dispenser within the interior space with a segment of the tab portion extending outside of the housing to prompt removal of the gas-impermeable membrane layer from the sealed position to the unsealed position by pulling the segment of the tab portion that is outside of the housing.

In another aspect, a dispenser for a scent-releasing apparatus having a housing is provided. The dispenser includes a blister and a gas-permeable membrane layer coupled to the blister to define a volume for containing a volatile liquid. The dispenser further includes a gas-impermeable membrane layer coupled to the gas-permeable membrane layer in a sealed position to substantially prevent ambient air from contacting the volatile liquid through the gas-permeable membrane layer. The gas-impermeable membrane layer includes a cover portion that substantially covers the gas-permeable membrane layer and a tab portion that extends from the cover portion. The tab portion is longer than the cover portion to facilitate the tab portion extending out of the housing to permit pulling the tab portion from outside of the housing to prompt removal of the gas-impermeable membrane layer from the sealed position to an unsealed position in which the volatile liquid is exposed to ambient air.

### BRIEF DESCRIPTION OF THE DRAWINGS

FIG. 1 is a perspective view of a scent-releasing apparatus;

FIG. 2 is an exploded view of the scent-releasing apparatus shown in FIG. 1;

FIG. 3 is a front view of a cap and a hanger of the scent-releasing apparatus shown in FIG. 1;

FIG. 4 is a front view of a base of the scent-releasing apparatus shown in FIG. 1;

FIG. 5 is a side view of a dispenser of the scent-releasing apparatus shown in FIG. 1;

FIG. 6 is an enlarged portion of the dispenser shown in FIG. 5;

FIG. 7 is another enlarged portion of the dispenser shown in FIGS. 5; and

FIG. 8 is a partial sectional view of the scent-releasing apparatus shown in FIG. 1.

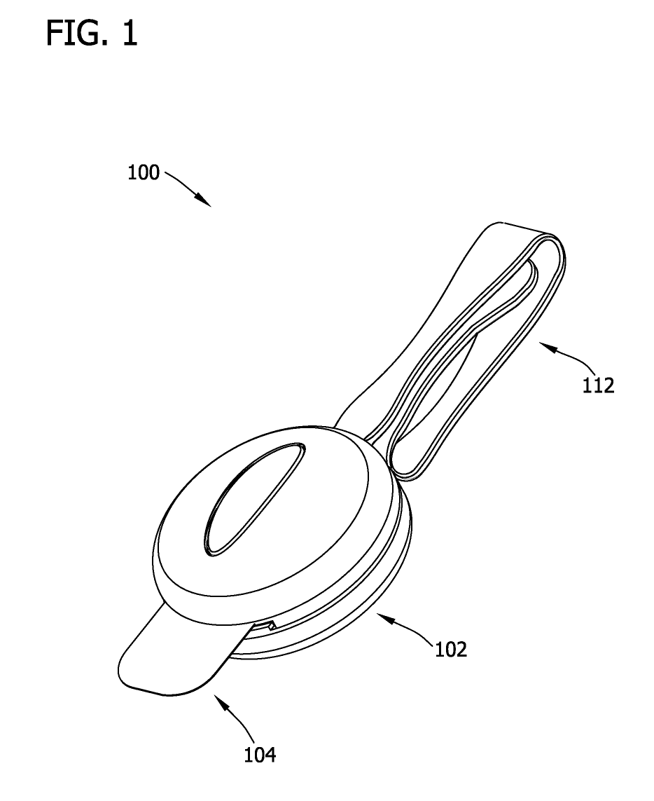

### DETAILED DESCRIPTION OF THE INVENTION

The exemplary embodiments described herein provide a scent-releasing apparatus for deodorizing a space and a method for making the same. The scent-releasing apparatus includes a housing and a dispenser disposed within the housing. The housing includes a base, a cap detachably coupled to the base, and a hanger integrally formed with and extending from the cap. The base includes a plurality of apertures that enable ambient airflow into the housing. The dispenser is disposed within the housing between the base and the cap, and the dispenser contains a scent-releasing material (e.g., a volatile liquid) that vaporizes when contacted by the ambient air to release a deodorizing scent into a space. The dispenser includes a transparent blister, a gas-permeable membrane layer coupled to the blister, and a gas-impermeable membrane layer detachably coupled to the blister over the gas-permeable membrane layer to facilitate preventing premature vaporization of the liquid (e.g., during shipment or storage of the scent-releasing apparatus).

To deploy the scent-releasing apparatus, a user grasps a tab of the gas-impermeable membrane layer that extends outside of the housing, and pulls the tab to peel at least a portion of the gas-impermeable membrane layer away from the gas-permeable membrane layer, thereby exposing the volatile liquid to the ambient air flowing into the housing through the apertures. The ambient air causes the volatile liquid to vaporize, which then releases a deodorizing scent from the housing. Once the apparatus is deployed and a deodorizing scent is being released from the housing, the apparatus can be placed (e.g., via the hanger) at a location that the user desires to deodorize, such as, for example. a restroom or a basement. Because the housing has a window, the user is permitted to view the amount of liquid remaining in the dispenser, enabling the user to gauge when the dispenser needs to be replaced. Also, because the user can deploy the scent-releasing apparatus by simply pulling a tab that extends outside of the housing, the user is not required to handle the dispenser in such close proximity of the scent-releasing material during deployment.

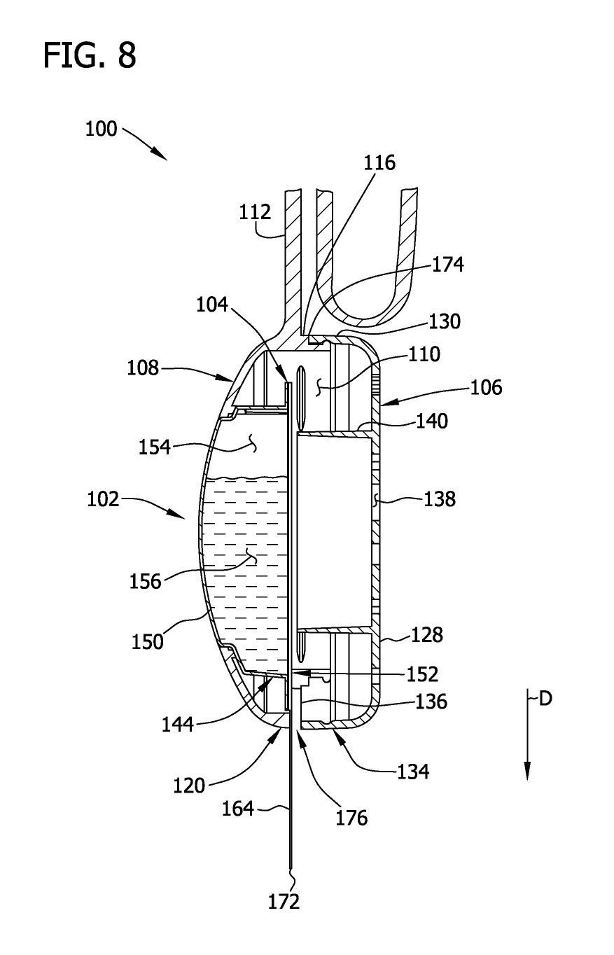

Figures I and 2 are perspective and exploded views, respectively, of an exemplary scent-releasing apparatus 100. Scent-releasing apparatus 100 includes a housing 102 and a dispenser 104 disposed within housing 102. Housing 102 includes a base 106 and a cap 108 connected to base 106 to define an interior space 110 (FIG. 8) of housing 102. A hanger 112 is integrally formed with and extends from cap 108 to facilitate mounting scent-releasing apparatus 100 after scent-releasing apparatus 100 has been deployed (i.e., when scent-releasing apparatus 100 is releasing a scent), as described below. In other embodiments, housing 102 may include any suitable number of components arranged in any suitable manner, and scent-releasing apparatus 100 may include any suitable mounting mechanism in addition to, or in lieu of, hanger 112.

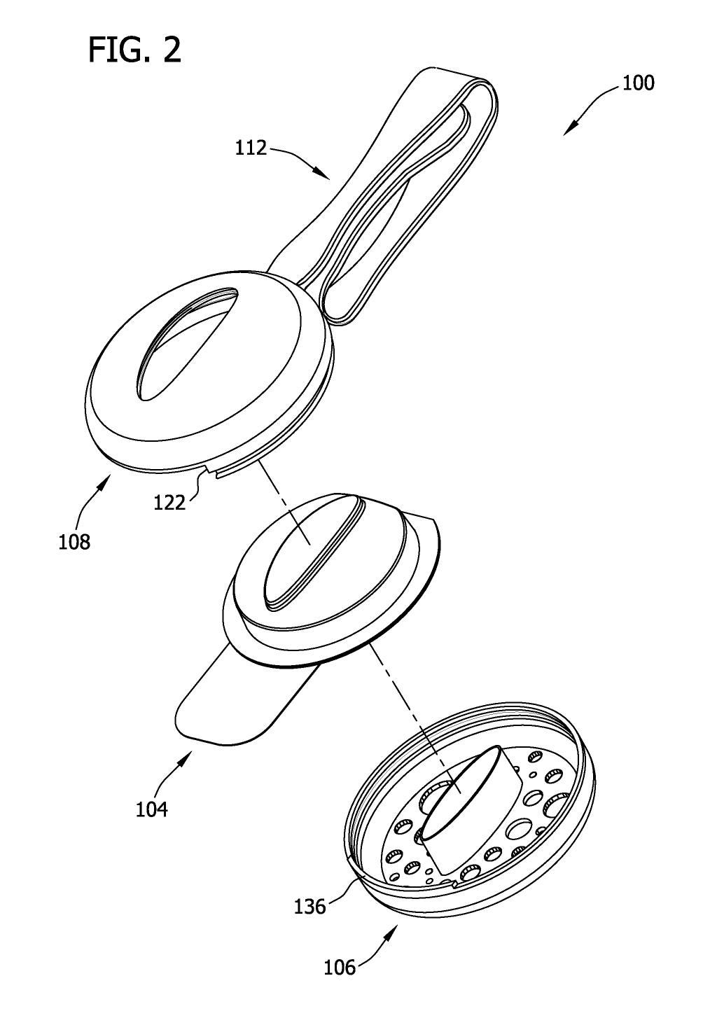

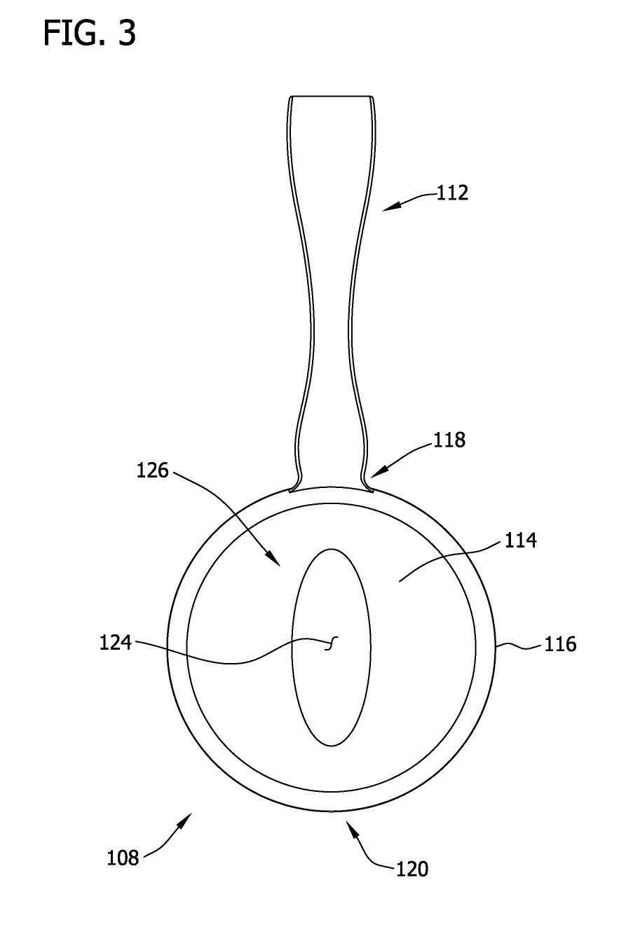

FIG. 3 is top view of cap 108 and hanger 112. In the exemplary embodiment, cap 108 has a top wall 114 and an annular side wall 116 extending from a periphery of top wall 114 such that cap 108 is generally dome-shaped (FIG. 2). Side wall 116 has an upper segment 118 and a lower segment 120, and hanger 112 extends from upper segment 118. A notch 122 (FIG. 2) is defined in lower segment 120, and a window 124 is defined in a central region 126 of top wall 114. Window 124 is elongated in a direction from upper segment 118 to lower segment 120 of side wall 116. In other embodiments, window 124 may have any suitable shape oriented in any suitable manner. In the exemplary embodiment, hanger 112 has a generally coiled formation (FIG. 2) and is resiliently flexible to facilitate at least partially unwrapping the coiled formation such that hanger 112 can be used to mount housing 102 in a wider variety of locations. Alternatively, hanger 112 may have any suitable configuration (e.g., hanger 112 may not be coiled and/or may not be flexible). In the exemplary embodiment, housing 102 and hanger 112 are integrally formed together from a synthetic or semi-synthetic. organic-based material (e.g., a “plastic” material) using a molding process. In one embodiment, housing 102 and hanger 112 may be fabricated from a polypropylene material. In other embodiments, housing 102 and/or hanger 112 may be fabricated from any suitable material.

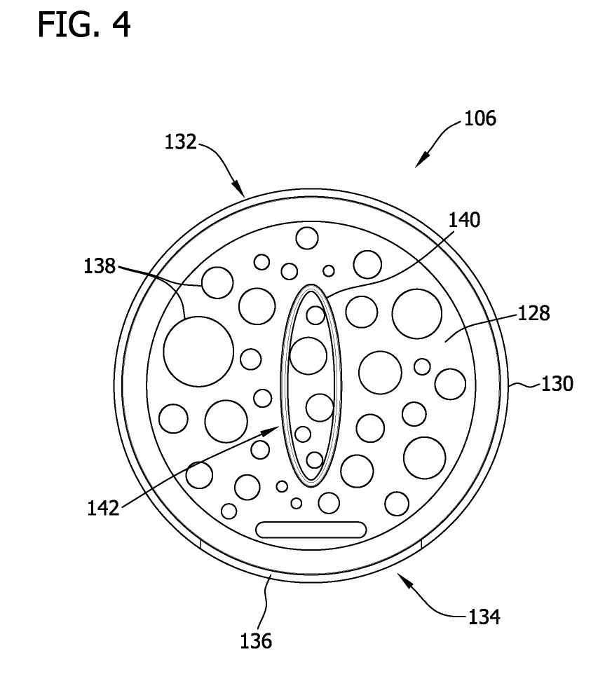

FIG. 4 is a top view of base 106. In the exemplary embodiment, base 106 has a bottom wall 128 and an annular side wall 130 extending from a periphery of bottom wall 128 such that base 106 is generally cup-shaped (FIG. 2). Side wall 130 has an upper segment 132 and a lower segment 134, and a step 136 is formed in side wall 130 at lower segment 134. Step 136 is sized for insertion into notch 122 of side wall 116 when cap 108 and base 106 are coupled together, as described below. In the exemplary embodiment, bottom wall 128 has a plurality of apertures 138 that are generally circular and vary in size. In other embodiments, cap 108 may include apertures 138, and/or apertures 138 may have any suitable shapes and sizes. In the exemplary embodiment, a hollow spacer. 140 extends into cup-shaped base 106 from a central region 142 of bottom wall 128, and spacer 140 is elongated in a direction from upper segment 132 to lower segment 134 of side wall 130. Alternatively, spacer 140 may not be hollow, and/or spacer 140 may have any suitable shape and disposition on bottom wall 128.

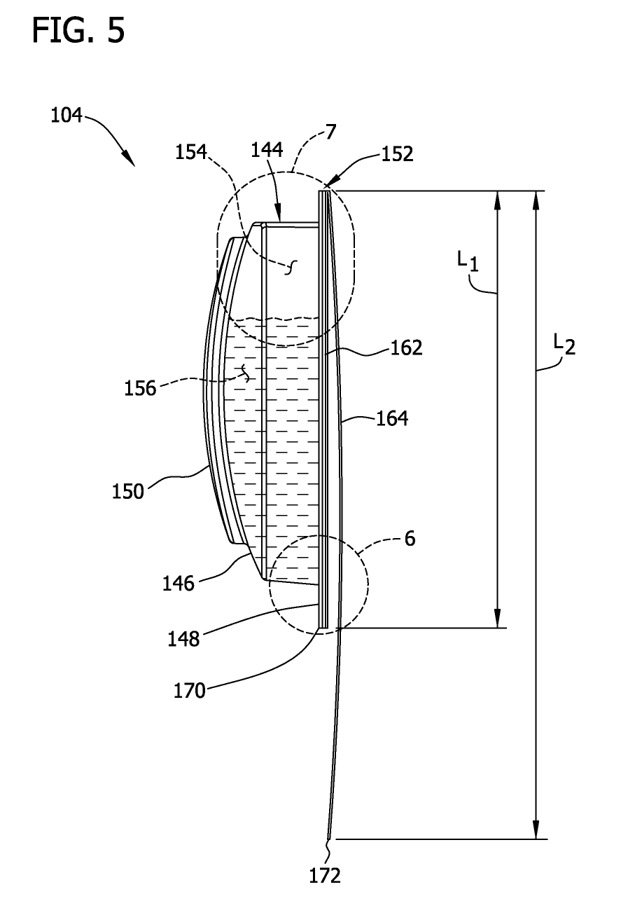

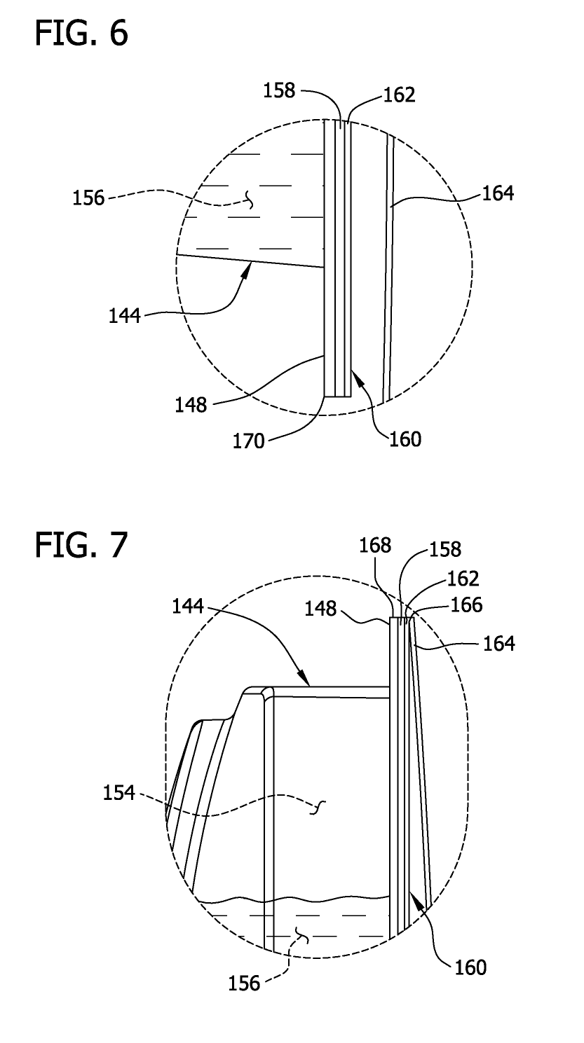

FIG. 5 is a side view of dispenser 104, and FIGS. 6 and 7 are enlarged portions of dispenser 104 shown in FIG. 5. In the exemplary embodiment, dispenser 104 includes a blister 144 having a top wall 146 and a flange 148 extending from top wall 146. Top wall 146 is generally dome-shaped to substantially match the dome-shape of cap 108 (e.g., top wall 146 includes a lip 150 sized for insertion into window 124 of cap 108) such that at least a portion of top wall 146 is configured to be seated against cap 108 when dispenser 104 is inserted into cap 108, as described below. In the exemplary embodiment, top wall 146 and flange 148 are integrally formed together from any suitable synthetic or semi-synthetic, organic-based material (e.g., a “plastic” material) using a molding process. In one embodiment, blister 144 is rigid and is at least partially transparent or translucent. In other embodiments, blister 144 may not be rigid and/or may not be at least partially transparent or translucent.

In the exemplary embodiment, dispenser 104 further includes an arrangement 152 of layered materials coupled to flange 148 to define a volume 154 in which a scent-releasing material is contained. In the exemplary embodiment, the scent-releasing material is a volatile liquid 156 that vaporizes in ambient air to release a deodorizing scent. In other embodiments. the scent-releasing material may be any suitable material that is configured to release a scent for any suitable purpose (e.g., a solid material that sublimes in ambient air to release an insect-repelling scent). In the exemplary embodiment. arrangement 152 of layered materials includes a gas-permeable membrane layer 158 and a gas-impermeable membrane layer 160. Gas-permeable membrane layer 158 is coupled to flange 148 (e.g., via an adhesive) to partially define volume 154 and contain volatile liquid 156 within volume 154, and gas-impermeable membrane layer 160 is detachably coupled to gas-permeable membrane layer 158 (e.g., via an adhesive) such that gas-impermeable membrane layer 160 can be at least partially removed (e.g., peeled way) from gas-permeable membrane layer 158 to expose gas-permeable membrane layer 158 to ambient air.

When gas-impermeable membrane layer 160 is coupled to gas-permeable membrane layer 158, volume 154 is sealed in an air-tight manner, thereby substantially preventing premature vaporization of volatile liquid 156 (i.e., substantially preventing ambient air from entering volume 154 during shipment and/or storage of scent-releasing apparatus 100). In the exemplary embodiment, gas-permeable membrane layer 158 is fabricated using a low density polyethylene (LDPE) material, and gas-impermeable membrane layer 160 is fabricated using an aluminum foil. Alternatively, gas-permeable membrane layer 158 and gas-impermeable membrane layer 160 may be fabricated from any suitable materials. As used herein, the term “air-tight” refers to preventing a substantial amount of ambient airflow into volume 154, thereby inhibiting ambient air from contacting the scent-releasing material (e.g., volatile liquid 156). Also, as used herein, the term “scent-releasing material” refers to any material that is configured to release a scent or odor for a purpose such as, for example, deodorizing, insect repelling, or insect exterminating.

In the exemplary embodiment, gas-impermeable membrane layer 160 has a cover portion 162 and a tab portion 164 extending from cover portion 162. Cover portion 162 is sized to substantially cover gas-permeable membrane layer 158 and seal volume 154 (i.e., cover portion 162 has a “sealed position” in which cover portion 162 substantially prevents ambient airflow into volume 154 through gas-permeable membrane layer 158). In the exemplary embodiment, tab portion 164 is integrally formed with cover portion 162. and tab portion 164 is folded over cover portion 162 at a fold line 166 (i.e., at an initial removal portion of gas-impermeable membrane layer 160) adjacent an upper edge 168 of flange 148, and tab portion 164 extends from fold line 166 downward over cover portion 162 and past a lower edge 170 of flange 148. In other words, cover portion 162 has a first length L₁, and tab portion 164 has a second length L₂ that is greater than first length L₁ such that tab portion 164 has a distal end 172 that is below lower edge 170 of flange 148. In alternative embodiments, tab portion 164 may not be integrally formed with cover portion 162 (e.g., tab portion 164 may be formed separately from and coupled to cover portion 162), and/or tab portion 164 may extend from any suitable segment of cover portion 162 in any suitable manner than facilitates enabling dispenser 104 to function as described herein.

FIG. 8 is a partial sectional view of scent-releasing apparatus 100 in its assembled configuration prior to deployment (e.g., during shipment and/or storage). In the exemplary embodiment, side wall 116 of cap 108 is detachably coupled to side wall 130 of base 106 at a seam 174 (e.g., via a snap-fit engagement) to define interior space 110 of housing 102. Dispenser 104 is positioned within interior space 110 with lip 150 of blister 144 inserted into window 124 such that dispenser 104 is spaced apart from lower segments 120, 134 of cap 108 and base 106, respectively. Furthermore, arrangement 152 of layered materials is positioned adjacent to spacer 140 such that spacer 140 prevents dispenser 104 from contacting bottom wall 128 of base 106 (i.e., such that spacer 140 prevents dispenser 104 from blocking apertures 138, thereby assuring ambient airflow into, and out of, housing 102 through apertures 138). Spacer 140 also facilitates stabilizing a position of dispenser 104 within interior space 110 (i.e., facilitates holding lip 150 within window 124 to maintain dispenser 104 in a spaced apart relationship relative to lower segments 120, 134). With cap 108 connected to base 106, step 136 of base 106 is inserted into notch 122 of cap 108 to define a slot 176 between lower segments 120, 134. A segment of tab portion 164 extends through slot 176 such that distal end 172 of tab portion 164 is outside of housing 102 with fold line 166 disposed opposite of slot 176 (i.e., with the initial removal portion of gas-impermeable membrane layer 160 disposed opposite of slot 176). In such a pre-deployed configuration, cover portion 162 is in the sealed position such that ambient air is substantially prevented from flowing into volume. 154 (i.e., such that premature vaporization of volatile liquid 156 is substantially prevented until a user desires to deploy scent-releasing apparatus 100).

To prompt deployment of scent-releasing apparatus 100, a user simply grasps tab portion 164 proximate distal end 172 and pulls tab portion 164 in a direction D to impart a shearing force to gas-impermeable membrane layer 160 proximate its initial removal portion (i.e., proximate fold line 166). Because tab portion 164 is integrally formed with cover portion 162 adjacent upper edge 168 of flange 148, cover portion 162 is at least partially unsealed (e.g., peeled away) from gas-permeable membrane layer 158 to expose at least a portion of gas-permeable membrane layer 158 and volatile liquid 156 to the ambient air within interior space 110. With at least a portion of gas-permeable membrane layer 158 exposed, cover portion 162 is in an “unsealed position.” and ambient air flowing into interior space 110 of housing 102 through apertures 138 is permitted to begin vaporizing volatile liquid 156 to release a deodorizing scent into the ambient air. After gas-permeable membrane layer 158 is exposed to ambient air, volatile liquid 156 gradually vaporizes over a predetermined period of time (e.g., a few months, weeks, or days), and the amount of volatile liquid 156 within volume 154 decreases until volume 154 is empty. Because blister 144 is at least partially transparent or translucent, the user can view the amount of volatile liquid 156 that remains in volume 154 while scent-releasing apparatus 100 is deployed, thereby enabling the user to gauge when dispenser 104 needs replacement (e.g., when volume 154 is substantially empty). Moreover, because the entire dispenser 104 (other than the segment of tab portion 164 that extends through slot 176) is enveloped by housing 102, the user can deploy scent-releasing apparatus 100 by simply grasping housing 102 in one hand and pulling on the segment of tab portion 164 that is outside of housing 102 with the other hand. Thus, scent-releasing apparatus 100 can be deployed without the user having to handle dispenser 104 in close proximity of volatile liquid 156 and/or gas-permeable membrane layer 158.

To replace an empty dispenser 104, a user simply detaches cap 108 from base 106, and the user removes and discards the empty dispenser 104. The user can then place bottom wall 128 of base 106 on a surface (e.g., a substantially flat surface such as a table) and place a new dispenser 104 (i.e., a dispenser 104 having the air-tight seal of volume 154 intact, as described above) on spacer 140 such that arrangement 152 of layered materials is seated against spacer 140 with a segment of tab portion 164 extending over and resting on step 136. With the new dispenser 104 placed on spacer 140, the user couples cap 108 to base 106 at seam 174 such that lip 150 of blister 144 extends into window 124 and such that tab portion 164 extends through slot 176 with distal end 172 outside of housing 102. To re-deploy scent-releasing apparatus 100, the user simply pulls tab portion 164 in direction D (as described above), and the newly deployed scent-releasing apparatus 100 can then be placed at a location that the user desires to deodorize.

The methods and systems described herein therefore facilitate providing a scent-releasing apparatus having a dispenser disposed within a housing and a spacer that maintains a position of the dispenser within the housing while ensuring ambient airflow into the housing. Also, the methods and systems described herein facilitate providing a scent-releasing apparatus having a housing with a window that enables a user to view a level of scent-releasing material remaining in the apparatus. Moreover, the methods and systems described herein facilitate providing a scent-releasing apparatus that can be easily deployed by simply pulling a tab that is outside of the housing, thereby preventing the user from having to handle the dispenser in close proximity of the scent-releasing material during deployment.

Exemplary embodiments of a scent-releasing apparatus are described above in detail. The scent-releasing apparatus described herein is not limited to the specific embodiments described herein, but rather, components of the apparatus may be utilized independently and separately from one another. For example, the apparatus described herein may have other applications not limited to deodorizing, as described herein. Rather, the apparatus described herein can be implemented and utilized in connection with various other industries.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A scent-releasing apparatus comprising:
a housing comprising a base and a cap coupled to said base to define an interior space, wherein at least one of said base and said cap comprises a plurality of apertures for permitting ambient airflow into said interior space; and
a dispenser positioned within said housing, said dispenser comprising a blister and a gas-impermeable membrane layer removably coupled to said blister for covering a volume for containing a scent-releasing material, said gas-impermeable membrane layer configured to substantially seal the scent-releasing material from ambient air when in a sealed position, said gas-impermeable membrane layer comprising a tab portion extending at least partially outside of said housing, said tab portion configured to prompt removal of said gas-impermeable membrane layer from the sealed position to an unsealed position in which the scent-releasing material is exposed to ambient air.

2. A scent-releasing apparatus in accordance with claim 1, wherein said dispenser further comprises a gas-permeable membrane layer to which said gas-impermeable membrane layer is coupled and from which said gas-impermeable membrane layer is at least partially removable.

3. A scent-releasing apparatus in accordance with claim 2, wherein said scent-releasing material is a volatile liquid that releases a deodorizing scent when contacted by ambient air.

4. A scent-releasing apparatus in accordance with claim 3, wherein said blister is at least partially translucent or transparent.

5. A scent-releasing apparatus in accordance with claim 4, wherein said cap comprises a window for permitting a user to view a level of said volatile liquid through said blister.

6. A scent-releasing apparatus in accordance with claim 1, wherein said housing further comprises a spacer configured to maintain a position of said dispenser within said interior space.

7. A scent-releasing apparatus in accordance with claim 1, wherein said apertures vary in size.

8. A scent-releasing apparatus in accordance with claim 1, wherein said gas-impermeable membrane layer comprises an initial removal portion and wherein said housing comprises a slot through which said tab portion extends, said dispenser disposed within said housing with said initial removal portion opposite said slot.

9. A scent-releasing apparatus in accordance with claim 1, wherein said housing comprises a hanger for mounting said scent-releasing apparatus.

10. A scent-releasing apparatus in accordance with claim 9, wherein said hanger has a generally coiled formation, said hanger being resiliently flexible and at least partially unwrappable from the coiled formation.

11. A housing for a scent-releasing apparatus having a dispenser that defines a volume for containing a scent-releasing material that is substantially sealed from ambient air by a gas-impermeable membrane layer that is removable from a sealed position to an unsealed position via a tab portion to expose the scent-releasing material to the ambient air and release a scent into the ambient air, said housing comprising:
a base; and
a cap configured to be coupled to said base to define an interior space of said housing, wherein at least one of said base and said cap comprises a plurality of apertures for permitting ambient airflow into said interior space, said housing configured to position the dispenser within said interior space with a segment of the tab portion extending outside of said housing to prompt removal of the gas-impermeable membrane layer from the sealed position to the unsealed position by pulling the segment of the tab portion that is outside of said housing.

12. A housing in accordance with claim 11, wherein said base and said cap are configured to be detachably coupled together to facilitate replacement of the dispenser.

13. A housing in accordance with claim 11, wherein said housing further comprises a spacer configured to maintain a position of the dispenser within said interior space.

14. A housing in accordance with claim 11, wherein said apertures vary in size.

15. A housing in accordance with claim 11, wherein said cap comprises a window configured to permit a user to view the scent-releasing material.

16. A dispenser for a scent-releasing apparatus having a housing, said dispenser comprising:
a blister;
a gas-permeable membrane layer coupled to said blister to define a volume for containing a volatile liquid; and
a gas-impermeable membrane layer coupled to said gas-permeable membrane layer in a sealed position to substantially prevent ambient air from contacting said volatile liquid through said gas-permeable membrane layer, said gas-impermeable membrane layer comprising a cover portion that substantially covers said gas-permeable membrane layer and a tab portion that extends from said cover portion, wherein said tab portion is longer than said cover portion to facilitate said tab portion extending out of the housing to permit pulling said tab portion from outside of the housing to prompt removal of said gas-impermeable membrane layer from the sealed position to an unsealed position in which the volatile liquid is exposed to ambient air.

17. A dispenser in accordance with claim 16. wherein said blister is at least partially translucent or transparent.

18. A dispenser in accordance with claim 16, wherein said blister comprises an annular flange to which said gas-permeable membrane layer is coupled.

19. A dispenser in accordance with claim 16, wherein said cover portion and said tab portion are integrally formed together from an aluminum foil material.

20. A dispenser in accordance with claim 19, wherein said tab portion is folded relative to said cover portion.
